(19) United States
(12) Patent Application Publication
(10) Pub. No.: US 2022/0403030 A1
(43) Pub. Date: Dec. 22, 2022

(54) COMPOSITION, COMPRISING COTL1 AS ACTIVE INGREDIENT, FOR DIAGNOSIS OF BONE DISEASE OR OBESITY

(71) Applicant: AJOU UNIVERSITY INDUSTRY-ACADEMIC COOPERATION FOUNDATION, Suwon-si (KR)

(72) Inventors: Seon Yong JEONG, Yongin-si (KR); Eunkuk PARK, Suwon-si (KR); Changgun LEE, Suwon-si (KR)

(73) Assignee: AJOU UNIVERSITY INDUSTRY-ACADEMIC COOPERATION FOUNDATION, Suwon-si (KR)

(21) Appl. No.: 17/777,460

(22) PCT Filed: Nov. 19, 2020
(86) PCT No.: PCT/KR2020/016333

§ 371 Date: May 17, 2022

(30) Foreign Application Priority Data
Nov. 19, 2019 (KR) 10-2019-0149077
Nov. 19, 2019 (KR) 10-2019-0149078
Nov. 19, 2020 (KR) 10-2020-0155080
Nov. 19, 2020 (KR) 10-2020-0155081

(51) Int. Cl. C07K 16/28; C07K 14/47; A61P 3/04; A61P 19/08

(52) U.S. Cl. CPC C07K 16/2818; C07K 14/4702; A61P 3/04; A61P 19/08

(57) ABSTRACT

The present invention relates to a composition, comprising COTL1 as an active ingredient, for diagnosis of a bone disease or obesity. Downregulation of the expression of the COTL1 protein or a gene thereof inhibits the differentiation activity of osteoclasts to reinforce bone density, compared to a normal control, and to increase factors that induce articular inflammation and cartilage degeneration, causing a bone disease such as osteosclerosis or osteoarthritis. Thus, COTL1 protein or a gene coding therefor can be used for diagnosing, preventing, or treating a bone disease such as osteosclerosis or osteoarthritis. In addition, downregulation of the expression of COTL1 protein or a gene coding therefor exhibits an excellent effect of treating obesity by reducing body weight and body fat mass in high-fat diet obesity mouse models and suppressing hepatic fat accumulation and adipocyte sizes and can be used for diagnosing obesity. An inhibitor against the protein or a gene thereof can be used to effectively prevent, alleviate, or treat obesity.

### TECHNICAL FIELD

The present disclosure relates to a composition for diagnosing a bone disease or obesity including COTL1 as an active ingredient. More particularly, the present disclosure relates to a composition for diagnosing a bone disease or obesity including a COTL1 protein or a gene encoding the same, a composition for preventing or treating a bone disease, and a composition for preventing, alleviate, or treating obesity including a COTL1 inhibitor as an active ingredient.

### BACKGROUND ART

Osteoclasts, which are large multinucleated cells, have functions of destructing and resorbing bone tissues, and are known to play a role in destructing bone matrix and decomposing bone minerals. Activated osteoclasts have three or more nuclei, and various hormones and factors are required such as a macrophage colony stimulating factor (M-CSF) and receptor activator of a nuclear factor-kappa B ligand (RANKL) to differentiate into mature multinucleated osteoclasts from osteoclast progenitors.

The osteoclasts cause the destruction and resorption of abnormal bone tissues due to imbalance with osteoblasts in the bone, known to cause osteoporosis in which bone mass and bone density decrease, osteomalacia in which lime is lost from the bone, fibrous ostitis in which bone marrow becomes fibrous, periodontitis in which alveolar bone is lost, and rheumatoid arthritis that causes destruction and deformation of joints.

Thereamong, osteoarthritis is a disease mainly characterized by the loss of proteoglycan (PG), a component of articular cartilage, and is a major disease that causes the destruction of cells and constituent tissues in articular cartilage to cause joint dysfunction. When osteoarthritis occurs, the expression of MMP-3, MMP-9, and MMP-13 increases, and it is known that such increase in MMPs damages the collagen matrix constituting cartilage, thereby exacerbating degenerative arthritis.

Currently, non-steroidal anti-inflammatory drugs (NSAIDs), analgesics, hyaluronan, glucosamine, and chondroitin are mainly used as treatments for osteoarthritis, but theses only show symptom relief effects rather than fundamental treatment. Particularly, it is known that some NSAIDs inhibit the synthesis of proteoglycan in articular cartilage, which tends to worsen symptoms of osteoarthritis.

Therefore, there is an urgent need to develop an alternative method that can protect the articular cartilage itself or directly treat the damage.

Meanwhile, the World Health Organization (WHO) declared obesity a “disease that needs to be treated” and defined the same as a “new infectious disease of the 21^{st} century”. Obesity is determined by the body mass index (BMI) obtained by dividing the weight (kg) by the square of the height (m), and a BMI of 25 or higher is considered obese. As of 2014, it is estimated that 39% of the world's population over 18 years are obese or overweight. In particular, the obesity rate among children and adolescents has increased rapidly, and obesity among children and adolescents in the United States more than tripled from 5% to 16.9% between 1999 and 2008.

In Korea, according to the “2017 Obesity White Paper” published by the National Health Insurance Corporation, the obesity rate of 13.95 million people who took general health checkups and health checkups during the life transition period last year was 33.55%. Thereamong, there was a difference between men and women, with 41.29% of men and 23.74% of women. Only 29.99% of men were normal, with 25.64% overweight, 35.74% obese, 5.31% severely obese, and 0.24% extremely obese, representing that there were many people who were obese or more likely to be obese. Particularly, men in their 30 s with a BMI 25 or higher took 46.26%. On the other hand, the normal rate of women was 50.03% which is considered high, with 18.33% overweight, 19.54% obese, 3.59% severely obese, and 0.61% extremely obese. The severe obesity rate and extreme obesity rate were consistently higher for both men and women with lower incomes.

The national and social costs due to obesity are also considerable and increasing over time. According to the “Research Report on Improvement Plans for Insurers' Obesity Management Project for Improving Health Longevity” recently released by the Health Insurance Policy Research Institute under the National Health Insurance Corporation, the socio-economic cost due to obesity approximately doubled from 4.765 trillion won in 2006 to 9,150.6 billion won in 2015. The details of 9,150.6 billion won are 58.8% for medical expenses (5,381.2 billion won), 17.9% for premature death (1.63 trillion won), 14.9% for productivity loss (1.365.4 billion won), 5.3% for care expenses (486.4 billion won), and 3.1% for transportation costs (280.4 billion won).

Improvement in eating habits and regular exercise are required for prevention and treatment of obesity, but due to the nature of modern people, major efforts to control obesity are focused on anti-obesity drugs and health supplements. As interest in obesity is increasing worldwide, there is an urgent need to develop a therapeutic agent capable of diagnosing, preventing, or treating obesity more effectively.

### DISCLOSURE

### Technical Goals

It is an object of the present disclosure to provide a biomarker composition for diagnosing a bone disease or obesity using factors determined to be significantly related to the bone disease or obesity, a composition for diagnosing a bone disease or obesity, and a kit for diagnosing a bone disease or obesity including the same.

Another object of the present disclosure is to provide a method for providing information necessary for diagnosis of a bone disease or obesity by measuring the factors.

Another object of the present disclosure is to provide a composition for preventing, alleviating, or treating a bone disease including the factor or an activator thereof.

Another object of the present disclosure is to provide a composition for promoting osteoclast differentiation including the factor.

Another object of the present disclosure is to provide a composition for preventing, alleviating, or treating obesity including an inhibitor of the factor.

### Technical Solution

In order to achieve the above object, example embodiments of the present disclosure provide a biomarker composition for diagnosing osteosclerosis or osteoarthritis including a COTL1 protein or a gene encoding the same.

Example embodiments of the present disclosure provide a composition for diagnosing osteosclerosis or osteoarthritis including an agent for measuring the expression level of a COTL1 protein or a gene encoding the same, and a diagnostic kit for osteosclerosis or osteoarthritis including the same.

Example embodiments of the present disclosure provide a method for providing information necessary for diagnosing osteosclerosis or osteoarthritis, including measuring an expression level of a COTL1 protein or a gene encoding the same in a biological sample isolated from a subject.

Example embodiments of the present disclosure provide a pharmaceutical composition for preventing or treating osteosclerosis or osteoarthritis including a COTL1 protein or a gene encoding the same, an expression promoter or an activator thereof as an active ingredient, and a health functional food composition for preventing or alleviating osteosclerosis or osteoarthritis.

Example embodiments of the present disclosure provide a reagent composition for promoting osteoclast differentiation including a COTL1 protein or a gene encoding the same as an active ingredient.

In addition, example embodiments of the present disclosure provide a biomarker composition for diagnosing obesity including a COTL1 protein or a gene encoding the same.

Example embodiments of the present disclosure provide a composition for diagnosing obesity including an agent for measuring the expression level of a COTL1 protein or a gene encoding the same, and an obesity diagnostic kit including the same.

Example embodiments of the present disclosure provide a method for providing information necessary for diagnosing obesity, including measuring an expression level of a COTL1 protein or a gene encoding the same in a biological sample isolated from a subject.

Example embodiments of the present disclosure provide a pharmaceutical composition for preventing or treating obesity including a COTL1 inhibitor as an active ingredient, and a health functional food composition for preventing or alleviating obesity.

### Advantageous Effects

Inhibition of the expression of a COTL1 protein or a gene encoding the same according to example embodiments of the present disclosure suppresses the differentiation activity of osteoclasts to enhance bone density compared to a normal control group, and increases factors that induce joint inflammation and cartilage degeneration, thereby causing bone diseases such as osteosclerosis or osteoarthritis. Thus, the COTL1 protein or the gene encoding the same may be usefully applied to the diagnosis of such bone diseases.

In addition, by using the COTL1 protein or the gene encoding the same, or an expression promoter or activator thereof, a bone disease may be more effectively prevented, alleviated, or treated, and differentiation of osteoclasts may be promoted by using the COTL1 protein or the gene encoding the same.

In addition, the reduction in the expression of the COTL1 protein or the gene encoding the same according to example embodiments of the present disclosure has an effect of reducing body weight and body fat mass in a high-fat diet obese mouse model, and has excellent effects in alleviating obesity, such as suppression of hepatic fat accumulation and adipocyte size. Accordingly, the COTL1 protein or the gene encoding the same may be usefully applied to obesity diagnosis.

In addition, by applying an inhibitor of the COTL1 protein or the gene encoding the same, it is possible to more effectively prevent, alleviate, or treat obesity.

### BRIEF DESCRIPTIONS OF THE DRAWINGS

FIG. 1 shows confirmation of an osteoclast differentiation activity in COTL1 knock-out mice, wherein (a) shows changes in activity of an osteoclast differentiation marker, and (b) shows staining results thereof.

FIG. 2 shows results of observing changes in bone density of COTL1 knock-out mice and the like, wherein (a) shows a result of measuring bone density of COTL1 knock-out mice and normal mice, (b) shows micro-CT images of removed femoral regions, (c) is a result of numerical analysis of % bone volume (BV), (d) is a result of numerical analysis of trabecular thickness (Tb.Th), (e) is a result of numerical analysis of trabecular number (Tb.N), and (f) is a result of numerical analysis of trabecular spacing (Tb.Sp).

FIG. 3 shows observation of osteoclasts in the cartilage area of COTL1 knock-out mice and normal mice, wherein (a) shows confirmation by immunostaining and electron microscope (SEM), and (b) shows the number of stained cells.

FIG. 4 shows results of observing the expression of COX-2, MMP-3, and MMP-13, which are factors that induce joint inflammation and cartilage degeneration in osteoarthritis cell models of COTL1 knock-out mice and normal mice.

FIG. 5 shows changes in the body weight of COTL1 knock-out mice and normal mice fed with a high-fat diet for a total of 12 weeks.

FIG. 6 shows changes in the body fat in COTL1 knock-out mice and normal mice after 12 weeks of high-fat diet ingestion.

FIG. 7 shows observation of hepatic tissues and adipose tissues in COTL1 knock-out mice and normal mice after 12 weeks of ingestion of a normal diet and a high-fat diet through hematoxylin & eosin (H&E) staining.

FIG. 8 shows comparison of the size of adipocytes in COTL1 knock-out mice and normal mice after 12 weeks of ingestion of a normal diet and a high-fat diet.

### BEST MODE

Hereinafter, the present invention will be described in detail.

The present inventors determined that osteoclast differentiation activity was suppressed and factors that induce joint inflammation and cartilage degeneration were increased in COTL1 knock-out mice, and thus completed the present invention as a composition for diagnosis and treatment of bone diseases using the same.

In addition, the present inventors confirmed effects such as reducing body weight and body fat gain while suppressing fat accumulation and adipocyte size when COTL1 expression was knocked out in an obese mouse model fed with a high-fat diet, and thus completed the present invention as a composition for diagnosis and treatment of obesity using COTL1.

An example embodiment of the present disclosure provides a biomarker composition for diagnosing osteosclerosis or osteoarthritis including a COTL1 protein or a gene encoding the same.

In addition, an example embodiment of the present disclosure provides a biomarker composition for diagnosing obesity including a COTL1 protein or a gene encoding the same.

In the present specification, the term “COTL1” as used herein refers to a coactosin-like protein, whose gene encodes one of actin-binding proteins that regulate the actin cytoskeleton, and a protein thereof binds to F-actin and is stabilized by interacting with 5-lipoxygenase (ALOX5). It is also referred to as “coactosin-like F-actin binding protein” or “CLP” and may include a physiologically active fragment thereof having substantially the same activity as COTL1 or a fusion protein thereof, but is not limited thereto.

In the present specification, the term “osteosclerosis” as used herein refers to a disease in which most of the bone tissue is abnormally dense and the marrow space is narrowed and is also called Albers-Schonberg disease. When osteosclerosis occurs, the bones become hard, but rather brittle, so fractures occur easily. In addition, the spleen is enlarged, severe anemia occurs, the platelet count is reduced so that bleeding hardly stops, and the white blood cell count also decreases. However, there is no effective treatment yet.

In the specification, the term “osteoarthritis” as used herein refers to, as described above, a disease caused by damage to articular cartilage and tibial tissues underneath and is the most common form of arthritis. As a major degenerative disease, the joint becomes painful and stiff. At first, pain is felt only when moving, but pain continues as it becomes chronic.

In the present specification, the term “obesity” as used herein refers to a state in which adipose tissues are excessive in the body. As described above, it is considered obese if the body mass index, which is obtained by dividing the weight (kg) by the square of the height (m), is 25 or higher. Obesity may cause various complications, so symptoms may also occur thereby.

In the present specification, the term “diagnosis” as used herein refers to determining the presence or characteristics of a pathological state, and for the purpose of the present disclosure, determining the onset or progression of a bone disease, preferably the onset or progression of osteosclerosis or osteoarthritis. In addition, another purpose of the present disclosure may be to determine obesity.

In the present specification, the term “biomarker” as used herein refers to an indicator that can detect changes in the body, is a substance capable of checking the normal or pathological state of a living organism as well as changes thereof, and may include organic biomolecules such as polypeptides, nucleic acids, lipids, glycolipids, glycoproteins, and sugars (monosaccharides, disaccharides, and oligosaccharides). By using the same, it is possible to diagnose bone diseases such as osteosclerosis or osteoarthritis as in the present disclosure. In addition, obesity may be diagnosed as in the present disclosure.

In the biomarker according to an example embodiment of the present disclosure, when the expression of the COTL1 protein or the gene encoding the same is reduced, the differentiation activity of osteoclasts is suppressed to increase bone density, and expression of factors that induces joint inflammation and cartilage degeneration such as COX-2, MMP-3, and MMP-13 may be increased. Therefore, osteosclerosis or osteoarthritis may be diagnosed through the expression or activity changes of the protein or the gene thereof.

In addition, when the expression or activity of the COTL1 protein or the gene thereof is reduced, an increase in body weight or body fat is reduced, and an effect of alleviating obesity such as suppression of fat accumulation and adipocytes size may be derived. Therefore, obesity may be diagnosed through the expression or activity changes of the protein or the gene thereof.

The biomarker according to the example embodiment of the present disclosure shows the same result even in repeated experiments, and since the change in the expression level thereof shows a significant result, the biomarker may be considered as a highly reliable marker, and thus the predicted result thereby may be reasonably trusted.

An example embodiment of the present disclosure provides a composition for diagnosing osteosclerosis or osteoarthritis including an agent for measuring the expression level of a COTL1 protein or a gene encoding the same.

In addition, an example embodiment of the present disclosure provides a composition for diagnosing obesity including an agent for measuring the expression level of a COTL1 protein or a gene encoding the same.

The agent may be a primer or a probe that specifically binds to a COTL1 gene; or an antibody, a peptide, an aptamer, or a compound that specifically binds to the COTL1 protein, but is not limited thereto.

In the present specification, the term “primer” as used herein refers to a nucleic acid sequence having a short free 3′ hydroxyl group, meaning a short nucleic acid sequence capable of base pairing with a complementary template and serving as a starting point for template strand replication. The primer is capable of initiating DNA synthesis in the presence of a reagent for a polymerization reaction (e.g., DNA polymerase or reverse transcriptase) and four different nucleotide triphosphates at an appropriate buffer and temperature.

The primer is a primer specific for the gene, and may be sense and antisense nucleic acids, typically having a sequence of 7 to 50 nucleotides. As long as it does not change the basic properties of the primer serving as a starting point of DNA synthesis, additional characteristics may be combined. PCR conditions and lengths of sense and antisense primers may be appropriately selected according to techniques known in the art.

In the present specification, the term “probe” as used herein refers to a nucleic acid fragment such as RNA or DNA corresponding to several bases to several hundred bases in length with the ability to specifically bind to mRNA and is labeled so that the presence of a specific mRNA and expression level may be detected. The probe may be prepared in the form of oligonucleotide probes, single strand DNA probes, double strand DNA probes, or RNA probes. Appropriate probes and hybridization conditions may be appropriately selected according to techniques known in the art.

In the present specification, the term “antibody” as used herein is a term known in the art and refers to a specific immunoglobulin directed against an antigenic site. The antibody in the present disclosure refers to an antibody that specifically binds to the protein and may be prepared according to a conventional method in the art. The form of the antibody includes a polyclonal antibody or a monoclonal antibody, and any immunoglobulin antibody may be included. The antibody refers to a complete form having two full-length light chains and two full-length heavy chains. In addition, the antibody may include special antibodies such as a humanized antibody.

In the present specification, “peptide” has high binding strength to a target material and does not undergo denaturation even upon heat/chemical treatment. In addition, due to small molecular size, the peptide may be used as a fusion protein by attaching the same to other proteins. Specifically, the peptide may be used by being attached to a polymeric protein chain so as to be used as a diagnostic kit and a drug delivery material.

In the present specification, the term “aptamer” as used herein refers to a type of polynucleotides consisting of special type of single strand nucleic acids (DNA, RNA, or modified nucleic acid) having a stable tertiary structure by itself with ability to bind to a target molecule with high affinity and specificity. As described above, the aptamer is able to specifically bind to an antigenic substance as an antibody, has higher stability than a protein and a simple structure, and is formed of easily synthesizable polynucleotides, such that the aptamer may be used in replacement of an antibody.

In the diagnostic composition according to an example embodiment of the present disclosure, when the expression of the COTL1 protein or the gene encoding the same is reduced, the differentiation activity of osteoclasts is suppressed to increase bone density while the expression of factors that induce joint inflammation and cartilage degeneration such as COX-2, MMP-3, and MMP-13 may be increased, thereby diagnosing osteosclerosis or osteoarthritis by measuring the expression level of the COTL1 protein or the gene thereby using the agent.

In addition, when the expression of the COTL1 protein or the gene encoding the same is suppressed, the obesity alleviating effect may be confirmed, thereby diagnosing obesity by measuring the expression level of the protein or the gene thereof using the agent.

Corresponding features may be substituted for the above-mentioned parts.

An example embodiment of the present disclosure provides a diagnostic kit for osteosclerosis or osteoarthritis including the composition for diagnosing osteosclerosis or osteoarthritis.

In addition, an example embodiment of the present disclosure provides an obesity diagnostic kit including the composition for diagnosing obesity.

The kit may be a primer kit, a DNA chip kit, or a protein chip kit, but is not limited thereto. The kit may include an antibody that selectively recognizes a marker as well as a type or one or more other component compositions, solutions, or devices suitable for an analysis method, for the diagnosis of osteosclerosis or osteoarthritis, or obesity.

For example, the kit may include a substrate, a suitable buffer, a secondary antibody labeled with a chromogenic enzyme or a fluorescent substance, and a chromogenic substrate for immunological detection of the antibody. The substrate may include a nitrocellulose membrane, a 96-well plate synthesized with a polyvinyl resin, a 96-well plate synthesized with a polystyrene resin, and a slide glass made of glass, the chromogenic enzyme may include peroxidase and alkaline phosphatase, and FITC and RITC may be used as the fluorescent substance. In addition, as the chromogenic substrate solution, 2,2′-azino-bis(3-ethylbenzothiazoline-6-sulfonic acid) (ABTS), o-phenylene diamine (OPD), or tetramethyl benzidine (TMB) may be used.

Corresponding features may be substituted for the above-mentioned parts.

An example embodiment of the present disclosure provides a method for providing information necessary for diagnosing osteosclerosis or osteoarthritis including measuring an expression level of a COTL1 protein or a gene encoding the same in a biological sample isolated from a subject.

In addition, an example embodiment of the present disclosure provides a method for providing information necessary for diagnosing obesity including measuring an expression level of a COTL1 protein or a gene encoding the same in a biological sample isolated from a subject.

In the present specification, the term “subject” as used therein refers to an individual who is to determine the onset of osteosclerosis or osteoarthritis, or obesity or to predict the risk of the onset, wherein the type of the subject is not limited if the subject is an animal that can develop osteosclerosis or osteoarthritis, or obesity. Specifically, it may be a mammal, for example, a human (Homo sapiens).

The biological sample may be selected from the group consisting of blood, serum, serum-derived exosomes, tissues, urine, and saliva that the expression level of the protein or the gene thereof is different from that of a normal control group, but is not limited thereto.

When the expression level of the COTL1 protein or the gene encoding the same measured in the biological sample isolated from the subject is lower than that of the normal control group, osteosclerosis or osteoarthritis may be diagnosed.

In addition, when the expression level of the COTL1 protein or the gene encoding the same measured in the biological sample isolated from the subject is higher than the expression level of the normal control group, obesity may be diagnosed.

The expression level of the gene may be measured by at least one method selected from the group consisting of next generation sequencing (NGS), polymerase chain reaction (PCR), reverse transcription polymerase chain reaction (RT-PCR), real-time polymerase chain reaction (Real-time PCR), RNase protection assay (RPA), microarray, and northern blotting, but is not limited thereto.

In addition, the expression or activity level of the protein may be measured by at least one method selected from the group consisting of western blot, enzyme-linked immunosorbent assay (ELISA), radioimmunoassay (RIA), radioimmunodiffusion, Ouchterlony immunodiffusion method, rocket immunoelectrophoresis, immunohistostaining, immunoprecipitation assay, complement fixation assay, and flow cytometry (FACS), but is not limited thereto.

An example embodiment of the present disclosure provides a screening method for a therapeutic agent for osteosclerosis or osteoarthritis, including treating a biological sample isolated from a subject with a test substance, measuring an expression level of a COTL1 protein or a gene encoding the same in the sample treated with the test substance, and selecting a test substance when the expression level of the COTL1 protein or the gene thereof in the sample treated with the test substance is increased compared to a control group not treated with the test substance.

The screening method is a method of comparing the increase or decrease in the expression or activity of the COTL1 protein or the gene thereof in the presence or absence of a candidate substance for a therapeutic agent for bone disease such as osteosclerosis or osteoarthritis, and may be useful for screening an activator of the COTL1 protein or the gene thereof and an agent for alleviating or treating bone diseases.

That is, the substance that increases the expression level of the COTL1 protein or the gene thereof may be selected as a therapeutic agent for osteosclerosis or osteoarthritis.

In addition, an example embodiment of the present disclosure provides a screening method for a therapeutic agent for obesity, including treating a biological sample isolated from a subject with a test substance, measuring an expression level of a COTL1 protein or a gene encoding the same in the sample treated with the test substance, and selecting a test substance when the expression level of the COTL1 protein or the gene thereof in the sample treated with the test substance is reduced compared to a control group not treated with the test substance.

The screening method is a method of comparing the increase or decrease in the expression or activity of the COTL1 protein or the gene thereof in the presence or absence of a candidate substance for a therapeutic agent for obesity, and thus may be useful for screening an inhibitor of the COTL1 protein or the gene thereof and an agent for alleviating or treating obesity.

That is, a substance that reduces the expression level of the COTL1 protein or the gene thereof may be selected as a therapeutic agent for obesity.

Corresponding features may be substituted for the above-mentioned parts.

An example embodiment of the present disclosure provides a pharmaceutical composition for preventing or treating osteosclerosis or osteoarthritis including a COTL1 protein or a gene encoding the same, an expression promoter or an activator thereof as an active ingredient.

The expression promoter or activator may be a known expression promoter or activator of the COTL1 protein or the gene thereof, but is not limited thereto. In addition, all substances capable of directly or indirectly enhancing the expression or activity of the COTL1 protein or the gene thereof may be included.

By enhancing the expression or activity of the COTL1 protein or the gene thereof, osteosclerosis or osteoarthritis may be prevented or treated.

In addition, an example embodiment of the present disclosure provides a pharmaceutical composition for preventing or treating obesity including a COTL1 inhibitor as an active ingredient.

The COTL1 inhibitor is an agent that inhibits the expression or activity of the COTL1 protein or the gene encoding the same, and thus it is possible to prevent or treat obesity by inhibiting the same.

The inhibitor may be selected from the group consisting of antisense nucleotides complementarily binding to COTL1 mRNA, small interfering RNA (siRNA), and short hairpin RNA (shRNA), or may be selected from the group consisting of antibodies, peptides, aptamers, compounds, and natural products specifically binding to COTL1, but is not limited thereto.

The pharmaceutical composition according to an example embodiment of the present disclosure may be prepared according to a conventional method in the pharmaceutical field. The pharmaceutical composition may be combined with a pharmaceutically acceptable, suitable carrier depending on the formulation, and if necessary, be prepared by further including excipients, diluents, dispersants, emulsifiers, buffers, stabilizers, binders, disintegrants, and solvents. The appropriate carrier does not degrade the activity or properties of the COTL1 protein or the gene encoding the same, the expression promoter or the activator thereof according to an example embodiment of the present disclosure, or an inhibitor, and may be selected differently depending on the dosage form and formulation.

In the present specification, the term “pharmaceutically acceptable” as used herein refers to that there is no toxicity to cells or humans exposed to the composition.

The pharmaceutical composition according to example embodiments of the present disclosure may be applied in any formulation, and more specifically, may be used by formulating the same into oral dosage forms, external preparations, suppositories, and parenteral dosage forms of sterile injection solutions according to conventional methods.

A solid formulation among the oral dosage forms is in the form of tablets, pills, powder, granules, and capsules and may be prepared by mixing at least one excipient, for example, starch, calcium carbonate, sucrose, lactose, sorbitol, mannitol, cellulose, and gelatin. In addition to simple excipients, lubricants such as magnesium stearate and talc may be included. In addition, the capsule formulation may further include a liquid carrier such as fatty oil in addition to the above-mentioned substances.

Among the oral dosage forms, liquid formulations include suspensions, liquid solutions, emulsions, and syrups. In addition to water and liquid paraffin which are commonly used simple diluents, various excipients, for example, wetting agents, sweeteners, fragrances, and preservatives may be included.

The parenteral formulation may include sterile aqueous solutions, non-aqueous solutions, suspensions, emulsions, freeze-dried formulations, and suppositories. As the non-aqueous solvents and suspensions, propylene glycol, polyethylene glycol, vegetable oils such as olive oil, and injectable esters such as ethyl oleate may be used. As a base of the suppositories, witepsol, macrogol, Tween 61, cacao butter, laurin fat, and glycerogelatin may be used. It is not limited thereto, and any suitable agent known in the art may be used.

In addition, in the pharmaceutical composition according to an example embodiment of the present disclosure, calcium or vitamins may further be added to enhance therapeutic efficacy.

In the pharmaceutical composition according to an example embodiment of the present disclosure, the pharmaceutical composition may be administered in a pharmaceutically effective amount.

In the present specification, the term “pharmaceutically effective amount” as used therein refers to an amount sufficient to treat a disease at a reasonable benefit/risk ratio applicable to medical treatment while not causing side effects.

The effective dose level of the pharmaceutical composition may be differently determined depending on the purpose of use, the age, sex, weight, and health status of a patient, the type of disease, the severity, the activity of a drug, the sensitivity to the drug, the administration method, the administration duration, the administration route and the excretion rate, the treatment duration, factors including drugs blended or used in combination with, and other factors well known in the medical field. For example, although not constant, generally 0.001 to 100 mg/kg, preferably 0.01 to 10 mg/kg, may be administered once to several times a day. The above dosage does not limit the scope of the present disclosure in any way.

The pharmaceutical composition according to an example embodiment of the present disclosure may be administered to any animal that can develop osteosclerosis or osteoarthritis, or obesity, and the animal is, for example, not only humans and primates but also livestock such as cattle, pigs, horses, and dogs.

The pharmaceutical composition according to an example embodiment of the present disclosure may be administered by an appropriate administration route depending on the form of the formulation, and may be administered via various routes, either oral or parenteral, as long as it can reach a target tissue. The administration method is not particularly limited and may be conducted in a conventional manner, for example oral, rectal or intravenous, muscle, skin application, respiratory inhalation, and intrauterine dural or intracere-broventricular injection.

The pharmaceutical composition according to an example embodiment of the present disclosure may be used alone for the prevention or treatment of osteosclerosis or osteoarthritis, or obesity, or may be used in combination with surgery or other drug treatment.

An example embodiment of the present disclosure provides a health functional food composition for preventing or alleviating osteosclerosis or osteoarthritis including a COTL1 protein or a gene encoding the same, an expression promoter or an activator thereof as an active ingredient.

In addition, an example embodiment of the present disclosure provides a health functional food composition for preventing or alleviating obesity including a COTL1 inhibitor as an active ingredient.

Corresponding features may be substituted for the above-mentioned parts.

In the health functional food composition according to an example embodiment of the present disclosure, the health functional food may be prepared in the form of powder, granules, tablets, capsules, syrups, or beverages for the purpose of preventing or alleviating osteosclerosis or osteoarthritis, or obesity. There is no limitation in the form that the health functional food may take, and the health functional food may be formulated in the same manner as the pharmaceutical composition so as to be used as a functional food or added to various foods.

In the health functional food composition according to an example embodiment of the present disclosure, the health functional food may include all foods in a conventional sense. For example, beverages and various drinks, fruits, and processed foods thereof (canned fruit and jam), fish, meat and processed foods thereof (ham and bacon), breads and noodles, cookies and snacks, and dairy products (butter and cheese) are possible, and all functional foods in a conventional sense may be included. In addition, food used as feed for animals may also be included.

The health functional food composition according to an example embodiment of the present disclosure may be prepared by further including food additives acceptable in food science and other suitable supplement ingredients commonly used in the art. The suitability as the food additive may be determined by the standards and criteria related to corresponding items according to the general rules and general test methods of Korean Food Additives Codex approved by the Ministry of Food and Drug Safety, unless otherwise stipulated. The items listed in the “Korean Food Additives Codex” may include, for example, chemical compounds such as ketones, glycine, calcium citrate, nicotinic acid, and cinnamic acid; natural additives such as persimmon pigments, licorice extracts, crystalline cellulose, kaoliang color, and guar gum; and mixed preparations such as sodium L-glutamate preparations, noodle-added alkali agents, preservative preparations, and tar color preparations.

The other auxiliary components may additionally include, for example, flavoring agents, natural carbohydrates, sweeteners, vitamins, electrolytes, coloring agents, pectic acid, alginic acid, organic acids, protective colloidal thickeners, pH adjusting agents, stabilizers, preservatives, glycerin, alcohols, and carbonating agents. In particular, monosaccharides such as glucose and fructose, disaccharides such as maltose and sucrose, polysaccharides such as dextrin and cyclodextrin, and sugar alcohols such as xylitol, sorbitol, and erythritol may be used as the natural carbohydrate, and natural sweeteners such as thaumatin and stevia extracts or synthetic sweeteners such as saccharin and aspartame may be used as the sweetener.

The effective dose of the COTL1 protein or the gene encoding the same, an expression promoter or an activator, or an inhibitor contained in the health functional food according to an example embodiment of the present disclosure may be appropriately adjusted depending on the purpose of use, such as prevention or alleviation of osteosclerosis or osteoarthritis, or obesity.

The health functional food composition uses food as a raw material, has the advantage of not having side effects that may occur when taking general drugs for a long time, and may be taken as an adjuvant for the prevention or alleviation of osteosclerosis or osteoarthritis, or obesity due to excellent portability.

In addition, an example embodiment of the present disclosure provides a reagent composition for promoting osteoclast differentiation including a COTL1 protein or a gene encoding the same as an active ingredient.

When the expression of the COTL1 protein or the gene encoding the same is suppressed, the differentiation activity of osteoclasts is suppressed. Thus, by increasing the expression of the protein or the gene thereof, it is possible to promote the differentiation of osteoclasts.

Corresponding features may be substituted for the above-mentioned parts.

Further, an example embodiment of the present disclosure provides a reagent composition for inhibiting the expression of one or more osteoarthritis inducing factors selected from the group consisting of COX-2, MMP-3, and MMP-13 including the COTL1 protein or the gene encoding the same as an active ingredient.

When the expression of the COTL1 protein or the gene encoding the same is suppressed, the expression of one or more osteoarthritis inducing factors selected from the group consisting of COX-2, MMP-3 and MMP-13 is increased. Thus, it is possible to suppress the expression of the factor by increasing the expression of the protein or the gene thereof.

Corresponding features may be substituted for the above-mentioned parts.

In addition, an example embodiment of the present disclosure provides a method for promoting osteoclast differentiation including treating an animal other than human with a COTL1 protein or a gene encoding the same, or an expression promoter or activator thereof.

Further, an example embodiment of the present disclosure provides a method for inhibiting expression of one or more osteoarthritis inducing factors selected from the group consisting of COX-2, MMP-3, and MMP-13, including treating cells isolated from an individual with a COTL1 protein or a gene encoding the same, or an expression promoter or activator thereof.

Corresponding features may be substituted for the above-mentioned parts.

### MODES FOR CARRYING OUT INVENTION

Hereinafter, examples will be described in detail to help the understanding of the present disclosure. However, the following examples are merely illustrative of the content of the present disclosure, and the scope of the present disclosure is not limited to the following examples. The examples of the present disclosure are provided to more completely explain the present disclosure to those skilled in the art.

1. Determination of Effects of COTL1 on Bone Disease

### <Example 1> Confirmation of Inhibition of Osteoclast Differentiation Activity in COTL1 Knock-Out Mice

Osteoclasts are derived from hematopoietic stem cells and play a role in bone resorption that destructs aged bones, and bone remodeling is maintained through the balanced action of bone formation by osteoblasts and bone resorption by osteoclasts.

In an attempt to confirm the inhibitory effect on osteoclast differentiation activity in COTL1 knock-out mice, the inventors of the present invention isolated mononuclear cells from femoral regions of mice, treated 30 ng/mL of a macrophage colony-stimulating factor (M-CSF) and 50 ng of a receptor activator of nuclear factor kappa-KB ligand (RANKL) together to induce differentiation for 3 days, and then measured tartrate-resistant acid phosphatase (TRAP) activity, which is a marker for osteoclast differentiation. At this stage, after washing the medium containing the cells with physiological saline, the TRAP activity was checked by measuring the absorbance at a wavelength of 405 nm using a Acid-Phosphatase Kit for the cells in the medium, and the osteoclasts were observed using a microscope after the TRAP staining.

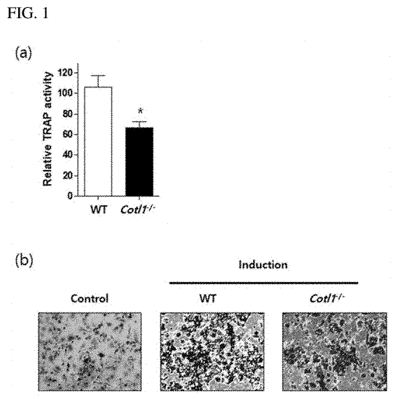

As a result, it was confirmed that osteoclasts isolated from COTL1 knock-out mice showed reduced TRAP activity compared to osteoclasts in normal mice, confirming that the osteoclast differentiation activity was significantly inhibited (FIG. 1).

### <Example 2> Determination of Bone Density in COTL1 Knock-Out Mice

Animal experiments for bone density were performed using 24-week-old female (C57BL6N) COTL1 knock-out mice and normal mice. At the start of the animal experiment, the initial bone mineral density (BMD) was measured with a PIXImus bone densitometer.

After anesthetizing mice by injecting 50 μl of an anesthetic mixed with zoletil and rompun (a 1:2 mixture was diluted with physiological saline at a 2:3 ratio), the mice were fixed in a bone density measuring frame to measure bone density. After termination of the experiment, the femur was removed to take micro-CT, and then % bone volume (BV), trabecular thickness (Tb.Th), trabecular number (Tb.N), and trabecular spacing (Tb.Sp) were numerically analyzed.

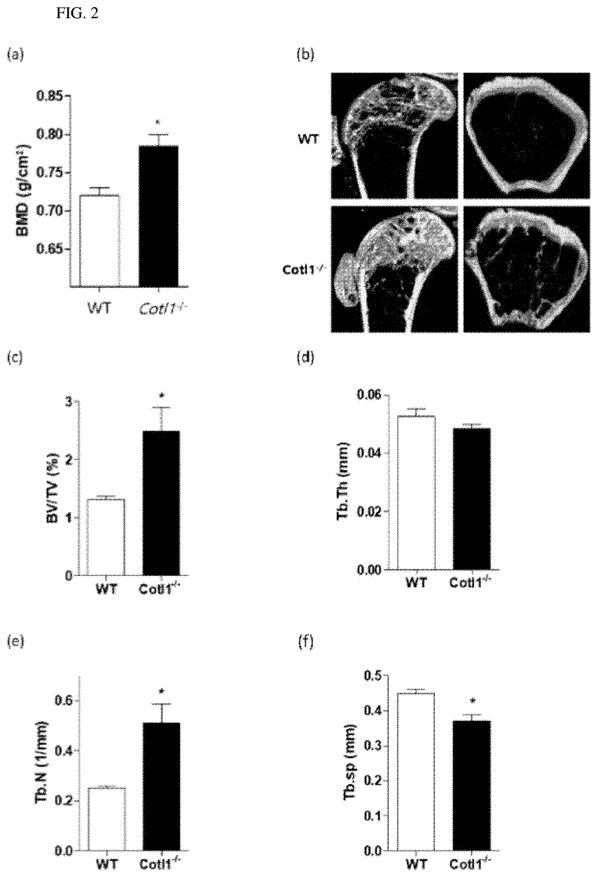

As a result, as shown in FIG. 2, bone mineral density (BMD) of COTL1 knock-out mice was increased compared to normal mice (FIG. 2a), and the density of bone microstructures on micro-CT images was increased (FIG. 2b). The % bone volume (FIG. 2c) and the trabecular number (FIG. 2e) were significantly increased, and the trabecular spacing (FIG. 2f) was found to be low.

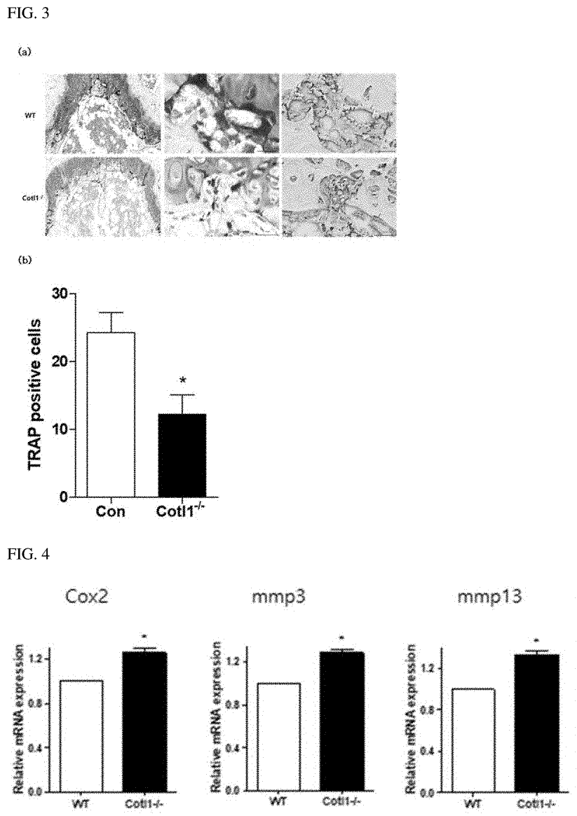

In addition, after analyzing the femur, the bone was sectioned, and TRAP immunostaining and electron microscopic (SEM) images, which are markers of osteoclast differentiation, were analyzed (FIG. 3).

### <Example 3> Cell Experiment to Determine the Osteoarthritis Alleviation Effect

Osteoarthritis (degenerative arthritis) is a disease in which the degeneration of articular cartilage tissues and structural changes in the bones under the cartilage occur, and the pathological cause of degenerative arthritis is not yet clearly found. The main causes include abrasion of cartilage due to repeated use of cartilage during the aging process, damage to cartilage tissues due to heredity and impact, pressure due to overweight, and muscle weakness around the knee. There are symptomatic treatments such as pain control and surgery, but a fundamental treatment has not yet been developed.

The inventors of the present invention isolated chondrocytes from the cartilage in 5-day-old mice, cultured the cells in a DMEM medium, and then prepared an osteoarticular cell model in order to determine the effect of inhibition of COTL1 expression on arthritis.

To verify the expression of catabolic factors (COX-2, MMP-3, and MMP-13) by inhibition of COTL1 expression at the cellular level, whether the catabolic factors secreted by chondrocytes were induced by inhibition of COTL1 expression was checked.

As a result, as shown in FIG. 4, it was confirmed that the expression of COX-2, MMP-3, and MMP-13, which are osteoarthritis inducing factors, was significantly increased when the expression of COTL1 was suppressed.

Based on these results, it was confirmed that COTL1 might be applied in pharmaceutical compositions for preventing, diagnosing, or treating bone diseases including osteosclerosis or osteoarthritis and screening methods for treating the diseases.

2. Determination of Effects of COTL1 on Obesity

### <Example 4> Confirmation of Suppression Efficacy for Body Weight and Body Fat in Obese Mouse Model

COTL1 knock-out mice and normal mice which are 4 weeks old were used. Experimental animals were bred in an animal breeding room at Ajou University, and after 7 days of adaptation to the environment, weight measurement and visual health condition check were carried out, and suitable mice were selected to be used for the experiment.

First, the feed provided to the mice to induce obesity was high-fat diet feed containing 60% fat from Research Diets (New Brunswick, N.J.), which was purchased from Dooyeol Bio, and the mice were allowed to freely take the feed with water for 8 weeks.

The dietary intake amount of the experimental animals was the same in the COTL1 knock-out mice and normal mice, the body weight was measured once a week from the start of the experiment, and the body fat was measured with a PIXImus bone densitometer after anesthetizing the mice by injecting 50 μl of a rompun-mixed anesthetic (1:2 mixed solution was diluted with physiological saline at a ratio of 2:3) on the last day of the experiment.

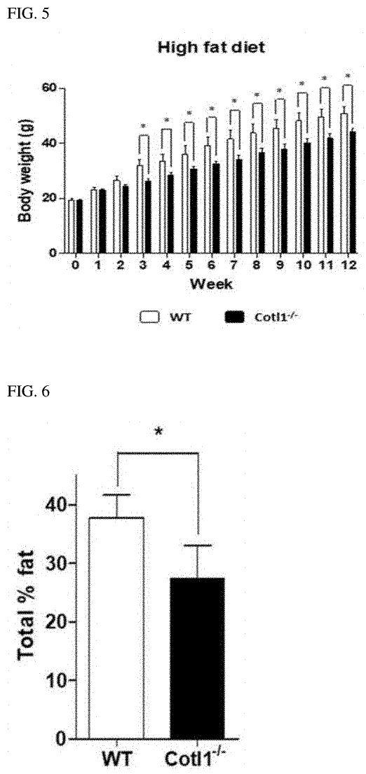

As a result, as shown in FIGS. 5 and 6, it was confirmed that the high-fat diet for 12 weeks showed a significant difference in body weight between the COTL1 knock-out mice and the normal mice from the 3^{rd} week of the experiment, and the body fat on the 8^{th} week was significantly reduced in the COTL1 knock-out mice.

### <Example 5> Confirmation of Efficacy in Inhibition of Hepatic Fat Accumulation and Adipocyte Size in an Obese Mouse Model

In order to confirm, in more detail, the effect of a high-fat diet on body weight and body fat suppression in COTL1 knock-out mice, the COTL1 knock-out mice and the normal mice were euthanized on the last day of the experiment after 12 weeks of the regular diet and the high-fat diet to remove the liver and abdominal fat of the mice, followed by fixation with 4% paraformaldehyde for 24 hours.

Thereafter, each tissue was prepared in a form of a paraffin block, tissue slides with a thickness of 3 μm were prepared using a microtome, and then hematoxylin & eosin (H&E) staining was performed. The stained slides were observed using a slide scanner. For the size of the observed abdominal fat, the length and width were measured using Caseviewer (Version. 1) software to measure the area.

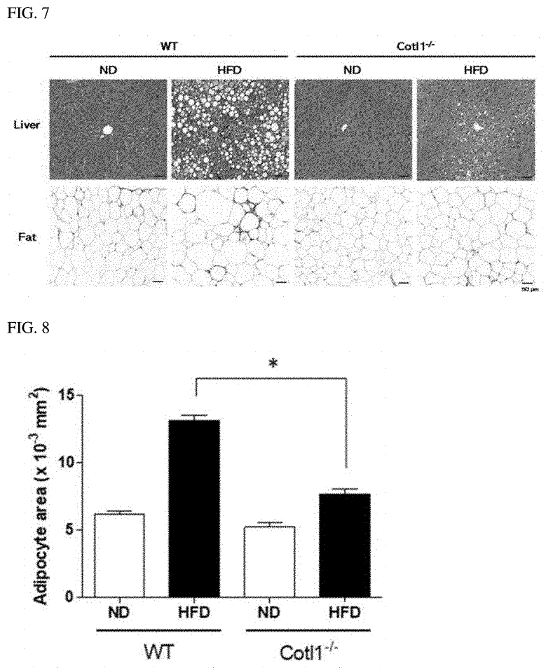

As a result, as shown in FIGS. 7 and 8, there was no significant difference in the generation of lipid droplets in the hepatic tissues of the normal mice and COTL1 knock-out mice fed with the regular diet for 12 weeks, but it was confirmed that the COTL1 knock-out mice fed with the high-fat diet feed for 12 weeks had significantly reduced hepatic lipid droplets and adipocyte size compared with the normal mice.

Through this, it was confirmed that body weight gain induced by the high-fat diet, the formation of hepatic lipid droplets, and the increase in the size of abdominal fat were suppressed in the COTL1 knock-out mice.

1-2. (canceled)

3. A composition for diagnosing osteosclerosis or osteoarthritis, comprising an agent for measuring an expression level of a COTL1 protein or a gene encoding the same.

4. The composition for diagnosing osteosclerosis or osteoarthritis of claim 3, wherein the agent is one of a primer or a probe that specifically binds to the COTL1 gene; or an antibody, a peptide, an aptamer, or a compound that specifically binds to the COTL1 protein.

5-8. (canceled)

9. A pharmaceutical composition for preventing or treating osteosclerosis or osteoarthritis, comprising a COTL1 protein or a gene encoding the same, or an expression promoter or activator thereof as an active ingredient.

10. A health functional food composition for preventing or alleviating osteosclerosis or osteoarthritis, comprising a COTL1 protein or a gene encoding the same, or an expression promoter or activator thereof as an active ingredient.

11-22. (canceled)
